(19) United States
(12) Patent Application Publication
(10) Pub. No.: US 2025/0134862 A1
(43) Pub. Date: May 1, 2025

(54) PROCESS FOR TREATING LIVESTOCK EXPOSED TO TOXIC FORAGE USING MELATONIN COMPOSITIONS

(71) Applicants: BOARD OF TRUSTEES OF THE UNIVERSITY OF ARKANSAS, Little Rock, AR (US); Mississippi State University, Starksville, MS (US)

(72) Inventors: Brittni P. Littlejohn, Farmington, AR (US); Caleb O. Lemley, Starkville, MS (US)

(73) Assignees: BOARD OF TRUSTEES OF THE UNIVERSITY OF ARKANSAS, Little Rock, AR (US); Mississippi State University, Starkville, MS (US)

(21) Appl. No.: 19/004,662

(22) Filed: Dec. 30, 2024

Related U.S. Application Data
(63) Continuation-in-part of application No. PCT/US23/69542, filed on Jun. 30, 2023.
(60) Provisional application No. 63/357,634, filed on Jun. 30, 2022.

(51) Int. Cl. A61K 31/4045; A61P 39/02

(52) U.S. Cl. CPC A61K 31/4045; A61P 39/02

(57) ABSTRACT

The invention relates to a process for treating livestock (e.g., gestating dams) exposed to toxic forage, such as endophyte-infected tall fescue or ryegrass, using a melatonin composition. The composition can include an active ingredient that includes melatonin having a concentration of up to about 250 μg/kg body weight of the livestock and an agriculturally acceptable carrier. The process can administer melatonin as a prophylactic or therapeutic for livestock consuming toxic forage or as a prophylactic or therapeutic for offspring of gestating dams that consume toxic forage.

### CROSS-REFERENCE TO RELATED APPLICATIONS

This application is a bypass continuation-in-part claiming priority to PCT/US2023/069542 filed on Jun. 30, 2023, which claims the benefit of U.S. Provisional Patent Application No. 63/357,634 filed on Jun. 30, 2022, and incorporates said applications by reference in their entirety into this document as if fully set out at this point.

### BACKGROUND OF THE INVENTION

### 1. Field of the Invention

This invention generally relates to a process for treating livestock exposed to toxic forage, such as endophyte-infected tall fescue or ryegrass, using melatonin compositions.

### 2. Description of the Related Art

Tall fescue is the most utilized cool-season forage present in the southeastern United States, making it an important and economically relevant forage to the region. Due to its vasoactivity, endophyte-infected tall fescue consumed during gestation has been thought to reduce blood flow to the uterus, potentially impacting the developing fetus.

Fescue toxicosis in cattle can result in decreased feed intake, decreased growth rates, inability to shed hair coats, inability to dissipate heat, decreased reproductive efficiency, decreased milk production, and damage to hooves, tail switches, and ear tips primarily due to severe vasoconstriction to the extremities. While additional research has characterized the impacts of fescue toxicosis on conception rates and pregnancy loss, limited results have been published on the health and performance of offspring from dams consuming endophyte-infected tall fescue (toxic fescue) during gestation. Recent findings showed that calves born to dams exposed to toxic fescue during gestation exhibited decreased birth weights and weaning weights. Other recent findings reported that differences exist in glucose homeostasis of calves born to dams that consumed toxic compared to novel fescue during gestation. Other evidence has demonstrated that toxic fescue consumption reduces uterine artery and vein areas in pregnant heifers, which is consistent with other recent findings demonstrating decreased uterine artery blood flow in pregnant heifers consuming toxic compared to endophyte-free fescue seed.

Melatonin is a neurohormone that is produced primarily by the pineal gland and has been shown to have effects on a myriad of physiological functions including blood pressure, immunomodulation, and circadian rhythms in mammals. Previous findings show melatonin supplementation increased uterine artery blood flow in a nutrient-restriction model in cattle. Furthermore, both serotonin (precursor hormone of melatonin) and melatonin have been found to be reduced in cattle consuming ergot alkaloids and endophyte-infected tall fescue seed, respectively.

While the negative effects of toxic fescue consumption on livestock have been long-established, an effective and cost-efficient therapeutic has not yet been identified and placed into practice. It is not logistically or economically practical for most producers to transition endophyte-infected tall fescue pastures to alternative forages.

### SUMMARY OF THE INVENTION

Accordingly, there is a critical need for a cost-effective process to mitigate the negative effects of 1) livestock grazing on toxic fescue and 2) toxic fescue consumed by gestating dams on offspring health and performance phenotypes.

It is, therefore, desirable to provide a novel process of using melatonin as a prophylactic or therapeutic for livestock exposed to toxic forage.

It is further desirable to provide a process of administering melatonin as a prophylactic or therapeutic for livestock exposed to endophyte-infected tall fescue or ryegrass or as a prenatal supplement to gestating dams consuming toxic forage for offspring health and performance phenotypes.

In general, in one aspect, the invention relates to a composition for treating livestock that consumes toxic forage, the composition comprising an active ingredient comprising melatonin having a concentration of up to about 250 μg/kg body weight (and any range or value therebetween) of the livestock and an agriculturally acceptable carrier.

In an embodiment, the melatonin has a concentration of about 50 μg/kg to about 200 μg/kg body weight (and any range or value therebetween) of the livestock.

In an embodiment, the melatonin has a concentration of about 100 μg/kg body weight (and any range or value therebetween) of the livestock.

In an embodiment, the livestock is selected from the group consisting of bovine/cattle, sheep, goats, lambs, pigs, horses, or poultry.

In an embodiment, the livestock comprises bovine that consumes toxic forage.

In an embodiment, the bovine is cattle.

In an embodiment, the cattle is a gestating dam that consumes toxic forage.

In an embodiment, the toxic forage is selected from the group consisting of endophyte-infected tall fescue or ryegrass.

In an embodiment, the toxic forage is endophyte-infected tall fescue.

In an embodiment, the carrier is selected from the group consisting of a veterinary composition, an animal supplement, or a food composition.

In an embodiment, the carrier is animal feed.

In an embodiment, the carrier is a transdermal implant.

In an embodiment, the carrier is a bolus dose.

In an embodiment, the livestock is cattle, the melatonin has a concentration of about 100 μg/kg body weight of the cattle, and the toxic forage is endophyte-infected tall fescue.

In general, in another aspect, the invention relates to a process for treating livestock that consumes toxic forage, the process comprising the steps of administering an active ingredient comprising melatonin having a concentration of up to about 250 μg/kg body weight (and any range or value therebetween) of the livestock and an agriculturally acceptable carrier to the livestock that consumes toxic forage.

In an embodiment, the process includes the step of administering the active ingredient comprising melatonin to the livestock as a prophylactic or therapeutic for the livestock that consumes toxic forage.

In an embodiment, the process includes the step of administering the active ingredient comprising melatonin to the livestock at a predetermined time of day as the prophylactic or therapeutic for the livestock that consumes toxic forage.

In an embodiment, the process includes the step of administering the active ingredient comprising melatonin to the livestock as a prophylactic or therapeutic for offspring of the livestock, where the livestock is a gestating dam that consumes toxic fescue.

In an embodiment, the process includes the step of administering the active ingredient comprising melatonin to the livestock at a predetermined time of day as the prophylactic or therapeutic for offspring of the livestock.

In an embodiment, the melatonin has a concentration of about 50 μg/kg to about 200 μg/kg body weight (and any range or value therebetween) of the livestock.

In an embodiment, the melatonin has a concentration of about 100 μg/kg body weight (and any range or value therebetween) of the livestock.

In an embodiment, the livestock is selected from the group consisting of bovine/cattle, sheep, goats, lambs, pigs, horses, or poultry.

In an embodiment, the livestock comprises bovine that consumes toxic forage.

In an embodiment, the bovine is cattle.

In an embodiment, the cattle is a gestating dam that consumes toxic forage.

In an embodiment, the toxic forage is selected from the group consisting of endophyte-infected tall fescue or ryegrass.

In an embodiment, the toxic forage is endophyte-infected tall fescue.

In an embodiment, the carrier is selected from the group consisting of a veterinary composition, an animal supplement, or a food composition.

In an embodiment, the carrier is animal feed.

In an embodiment, the carrier is a transdermal implant.

In an embodiment, the carrier is a bolus dose.

In an embodiment, the process includes the step of administering the active ingredient comprising melatonin having a concentration of about 100 μg/kg body weight (and any range or value therebetween) to the livestock, where the livestock is a gestating dam that consumes endophyte-infected tall fescue.

### BRIEF DESCRIPTION OF THE DRAWINGS

The implementations disclosed herein are illustrated by way of example, and not by way of limitation, in the figures of the accompanying drawings.

FIG. 1 is a top plan view of a barn at the Savoy Unit at the University of Arkansas with a controlled feeding system.

FIG. 2 is a flow chart of an example of a treatment protocol timeline in accordance with an illustrative embodiment of the invention disclosed herein, wherein AI: Artificial Insemination; SP: 70-d Dam Supplementation Period; BW: Birth Weight; WW: Weaning Weight; CU: Carcass Ultrasonography; WT: Calf Weight.

FIG. 3A is a graphical representation of the gestation length (d) of calves born to dams that were fed a diet consisting of endophyte-free fescue seed (green) and toxic fescue seed (red).

FIG. 3B is a graphical representation of the gestation length (d) of calves born to dams that were fed a diet consisting of melatonin (grey), and no melatonin (white).

FIG. 4 is a graphical representation of the gestation length (d) of calves born to dams that were fed a diet consisting of endophyte-free fescue seed with melatonin (green striped), endophyte-free fescue seed without melatonin (solid green), toxic fescue seed with melatonin (red striped), and toxic fescue seed without melatonin (red solid).

FIG. 5A is a graphical representation of the birth weight (BW) of calves born to dams that were fed a diet consisting of endophyte-free fescue seed (green) and toxic fescue seed (red).

FIG. 5B is a graphical representation of the birth weight of calves born to dams that were fed a diet consisting of melatonin (grey), and no melatonin (white).

FIG. 6 is a graphical representation of the birth weight of calves born to dams that were fed a diet consisting of endophyte-free fescue seed with melatonin (green striped), endophyte-free fescue seed without melatonin (solid green), toxic fescue seed with melatonin (red striped), and toxic fescue seed without melatonin (red solid).

FIG. 7A is a graphical representation of the 205-d adjusted weaning weight of calves born to dams that were fed a diet consisting of endophyte-free fescue seed with melatonin (green striped), endophyte-free fescue seed without melatonin (solid green), toxic fescue seed with melatonin (red striped), and toxic fescue seed without melatonin (red solid).

FIG. 7B is a graphical representation of the 205-d adjusted weaning weight of calves born to dams that were fed a diet consisting of endophyte-free fescue seed with melatonin (E−/M), endophyte-free fescue seed without melatonin (E−/NM), toxic fescue seed with melatonin (E+/M), and toxic fescue seed without melatonin (E+/NM).

FIG. 8 is a graphical representation of the calf ribeye area (REA) at weaning of calves born to dams that were fed a diet consisting of endophyte-free fescue seed with melatonin (green striped), endophyte-free fescue seed without melatonin (solid green), toxic fescue seed with melatonin (red striped), and toxic fescue seed without melatonin (red solid).

FIG. 9 is a graphical representation of calf rib area as a proportion of body weight (ribeye area per 100 lbs of body weight; REA/CWT) at weaning in calves born to dams that were fed a diet consisting of endophyte-free fescue seed with melatonin (green striped), endophyte-free fescue seed without melatonin (solid green), toxic fescue seed with melatonin (red striped), and toxic fescue seed without melatonin (red solid).

FIG. 10 is a graphical representation of the percent intramuscular fat (IMF) at weaning of calves born to dams that were fed a diet consisting of endophyte-free fescue seed with melatonin (green striped), endophyte-free fescue seed without melatonin (solid green), toxic fescue seed with melatonin (red striped), and toxic fescue seed without melatonin (red solid).

FIG. 11 is a graphical representation of the rib fat thickness (UFAT) at weaning of calves born to dams that were fed a diet consisting of endophyte-free fescue seed with melatonin (green striped), endophyte-free fescue seed without melatonin (solid green), toxic fescue seed with melatonin (red striped), and toxic fescue seed without melatonin (red solid).

FIG. 12 is a graphical representation of the rump fat thickness (URUMP) at weaning of calves born to dams that were fed a diet consisting of endophyte-free fescue seed with melatonin (green striped), endophyte-free fescue seed without melatonin (solid green), toxic fescue seed with melatonin (red striped), and toxic fescue seed without melatonin (red solid).

FIG. 13A is a graphical representation of the post-weaning calf body weight of calves born to dams that were fed a diet consisting of endophyte-free fescue seed (green) and toxic fescue seed (red).

FIG. 13B is a graphical representation of the post-weaning calf body weight of calves born to dams that were fed a diet consisting of melatonin (grey), and no melatonin (white).

FIG. 14 is a graphical representation of the post-weaning calf body weight of calves born to dams that were fed a diet consisting of endophyte-free fescue seed with melatonin (green striped), endophyte-free fescue seed without melatonin (solid green), toxic fescue seed with melatonin (red striped), and toxic fescue seed without melatonin (red solid).

FIG. 15A is a graphical representation over time of the post-weaning calf body weight of calves born to dams that were fed a diet consisting of endophyte-free fescue seed with melatonin (green stippled), endophyte-free fescue seed without melatonin (solid green), toxic fescue seed with melatonin (red stippled), and toxic fescue seed without melatonin (red solid).

FIG. 15B is a graphical representation over time of the post-weaning calf body weight of calves born to dams that were fed a diet consisting of endophyte-free fescue seed with melatonin (E−/M), endophyte-free fescue seed without melatonin (E−/NM), toxic fescue seed with melatonin (E+/M), and toxic fescue seed without melatonin (E+/NM) during mid-late gestation.

FIG. 16 is a flow chart of an example of a treatment protocol timeline in accordance with an illustrative embodiment of the invention disclosed herein, wherein AI: Artificial Insemination; WT: Dam Weight; U: Doppler Ultrasonography.

FIG. 17 is a graphical representation over time of the weights of dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 18 is a graphical representation of prolactin (ng/mL) for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 19 is a graphical representation of total uterine artery blood flow (mL/min) for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 20 is a graphical representation of total uterine artery blood flow (mL/min) as a proportion of body weight (kg) for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 21 is a graphical representation of ipsilateral uterine artery blood flow (mL/min) for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 22 is a graphical representation of ipsilateral uterine artery blood flow (mL/min) as a proportion of body weight (kg) for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 23 is a graphical representation of ipsilateral uterine artery pulsatility index for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 24 is a graphical representation of ipsilateral uterine artery resistance index for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 25 is a graphical representation of ipsilateral uterine artery diameter for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 26 is a graphical representation of coccygeal artery blood flow (mL/min) for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 27 is a graphical representation of coccygeal artery blood flow (mL/min) as a proportion of body weight (kg) for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 28 is a graphical representation of coccygeal artery pulsatility index for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 29 is a graphical representation of coccygeal artery resistance index for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 30 is a graphical representation of coccygeal artery diameter for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 31 is a flow chart of an example of a treatment protocol timeline in accordance with an illustrative embodiment of the invention disclosed herein, wherein WT: Dam & Calf Body Weight; MY: 4-h Milk Yield.

FIG. 32 is a graphical representation of the post-calving dam body weight (kg) for dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 33 is a graphical representation of the calf birth weight (kg) for calves born from dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 34A is a graphical representation of the calf body weight (kg) for calves born from dams that were fed a diet consisting of endophyte-free novel tall pastures and fed a supplement without melatonin (green), toxic tall fescue pastures and fed a supplement with melatonin (yellow), toxic tall fescue pastures and fed a supplement without meilatonin (red).

FIG. 34B is a graphical representation of the raw averages by treatment group for calf body weight (kg) of calves born from dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 35 is a graphical representation of the daily milk volume (L) estimate at d 28 from dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 36 is a graphical representation of the daily milk weight estimate at d 28 from dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 37 is a graphical representation of the daily milk volume estimate at d 56 from dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

FIG. 38 is a graphical representation of the daily milk weight estimate at d 56 from dams that were fed a diet consisting of endophyte-free novel tall pastures and fed a supplement without melatonin (green), toxic tall fescue pastures and fed a supplement with melatonin (yellow), toxic tall fescue pastures and fed a supplement without melatonin (red).

### DETAILED DESCRIPTION OF THE INVENTION

While this invention is susceptible of embodiment in many different forms, there is shown in the drawings, and will herein be described hereinafter in detail, some specific embodiments of the invention. It should be understood, however, that the present disclosure is to be considered an exemplification of the principles of the invention and is not intended to limit the invention to the specific embodiments so described.

The invention relates to a process for treating livestock exposed to toxic forage using melatonin compositions. The process can administer melatonin to livestock consuming toxic forage, such as at a predetermined time of day, as a prophylactic or therapeutic treatment for either the livestock or for their offspring (in the case of gestating dams consuming toxic forage) for increased health and performance phenotypes of the offspring. The inventive process can be used to treat farmed livestock, such as bovine/cattle, sheep, goats, lambs, pigs, horses, poultry, and the like. In addition to treating livestock that consumes endophyte-infected tall fescue, the inventive process can be utilized to treat livestock that consumes other types of toxic forages, such as ryegrass and other forages that contain a toxin-producing endophyte fungus, that have been associated with negative impacts on the performance of livestock.

Melatonin is incorporated into livestock management products (e.g., up to about 250 μg/kg of body weight, preferably about 50 μg/kg to about 200 μg/kg of body weight, preferably about 100 μg/kg to about 150 μg/kg of body weight, and more preferably about 100 μg/kg of body weight (and any range or value therebetween)) that can be administered during times of peak tall fescue growth. Melatonin can be delivered by methods such as diet, injectable, implant, transdermal, bolus dose, or other agriculturally acceptable carrier.

The melatonin composition, or functional/structural variant thereof, can be incorporated as an active ingredient in veterinary compositions, animal supplements, food compositions, or the like for preventing or treating various toxic forages in livestock. These compositions incorporating the melatonin composition may further contain protective hydrocolloids, such as gums, proteins, modified starches, binders, film-forming agents, encapsulating agents/materials, wall/shell materials, matrix compounds, coatings, emulsifiers, foaming agents, surface active agents, solubilizing agents, e.g., oils, fats, waxes, lecithin, etc., adsorbents, carriers, fillers, co-compounds, dispersing agents, wetting agents, processing aids (solvents), flowing agents, flavoring agents, sweetening agents, coloring agents, weighting agents, jellifying agents, gel-forming agents, anti-oxidants, anti-microbial and other preservative agents.

The veterinary compositions, animal supplements, or food compositions may be in any galenic formulation that is suitable for administrating to the livestock, especially in any form that is conventional for oral administration, e.g., in solid forms, such as (additives/supplements for) food or feed, food or feed premix, fortified food or feed, tablets, seed, pills, granules, capsules, or in liquid forms, such as solutions, emulsions or suspensions, e.g., liquids, pastes, and oily suspensions. The pastes may be filled into hard- or soft-shell capsules. Examples of other acceptable forms of administration are transdermal, parenteral, and injectable. The veterinary compositions, animal supplements, or food compositions may be in the form of controlled immediate or sustained-release formulations.

Melatonin is administered at predetermined times of the year when fescue toxicity is known to be an issue for livestock. Moreover, depending upon the delivery mode, melatonin can be administered dietarily at a predetermined time of the day to maintain melatonin concentrations that are physiologically similar to the livestock's normally elevated concentrations at night. For example, melatonin can be supplemented in the livestock's feed during a morning timeframe (e.g., between 9:00 am and 12:00 pm each day) to keep concentrations elevated to mimic nighttime concentrations during the day.

It is understood that the actual amount of the melatonin composition to be administered can vary in accordance with the age, size, condition, and other factors associated with the specific livestock to be treated, depending upon the discretion of the rancher or other producer.

### EXAMPLES

The process for treating livestock exposed to endophyte-infected tall fescue using melatonin compositions is further illustrated by the following examples, which are provided for the purpose of demonstration rather than limitation. In the following examples, melatonin compositions are evaluated as a prophylactic or therapeutic treatment to improve health, immune function, growth, performance, microbiome, metabolic function, feed efficiency, milk production, and reproductive traits in livestock that consumed toxic fescue and offspring of dams that consume toxic fescue during gestation.

### Example 1: Growth Performance and Estimates of Body Composition

In particular, the objective of this example was to evaluate growth performance and estimates of body composition in offspring of dams that consumed diets containing tall fescue seed with (E+) or without (E−) the toxic endophyte and with or without melatonin during mid-late gestation.

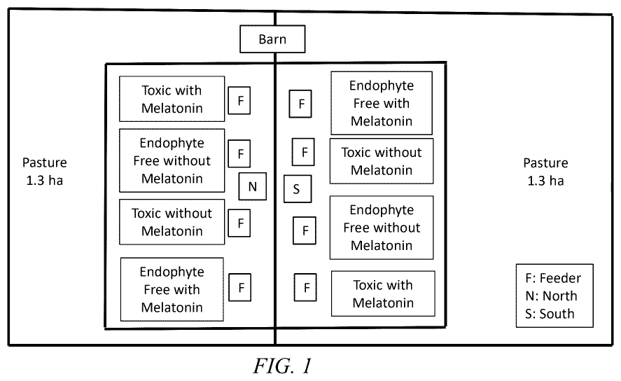

Fifty-seven (n=57) pregnant heifers artificially inseminated with sex-sorted semen (X chromosome-bearing sperm) from a single Angus sire were maintained at the Savoy Unit at the University of Arkansas on Bermuda grass pastures and supplemented as needed during the first 4-5 months of gestation. After that time, pregnant heifers were housed in a barn at the Savoy Unit (FIG. 1) having a controlled feeding system (SmartFeed Pro, C-Lock, Inc.). The controlled feeding system used radio frequency identification to control feed mixtures and intake for heifers.

Heifers were maintained in two groups (n=29 in each group), one group on the North side of the barn and the other on the South side of the barn (FIG. 1). Heifers underwent an approximate one-month training period to become acclimated to the feeding system, and the training period diet consisted of an ad libitum mixed grass hay (predominately Bermuda) and corn gluten pellets at about three (3) lbs./head/day.

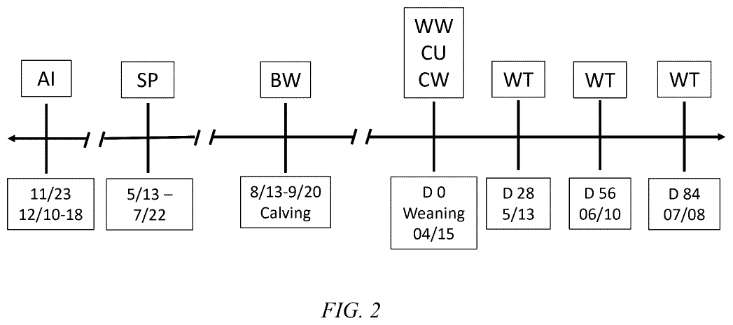

After the training period, the heifers started a 70-day feeding trial (FIG. 2) to evaluate the influence of toxic fescue and melatonin on the developing fetus. To mimic the natural peak of ergot alkaloids in Kentucky 31 fescue, toxic fescue seed was fed with or without melatonin during the 70-day period between May and July. The feed trial period diet consisted of an ad libitum Bermuda grass hay and one of four (4) feed rations. The trial utilized a 2×2 factorial treatment arrangement (E− without melatonin, E−/NM; E− with melatonin, E−/M; E+ without melatonin, E+/NM; or E+ with melatonin, E+/M; melatonin dose: 100 μg/kg of BW) for a 70-d period between May and July starting at 160±13 days of gestation to mimic ergot alkaloid concentrations (ergot alkaloid dose: 13 μg/kg of BW) in a grazing scenario (FIG. 2). Each of the four feed ration treatments was represented in each of the two pens/groups of cattle (FIG. 1). All diets contained corn gluten and molasses and were fed using SmartFeed Pro (C-Lock Inc.) controlled feeding units between 0900 and 1400 h daily. Cattle were removed from dietary treatments 39±19 days before calving. Two replicates were housed separately in the open-air barn (FIG. 1), each with a 1.2-ha paddock. Before and following the 70-d treatment period, all cattle were maintained as a single group free from toxic fescue.

The heifers were all fitted with RFID ear tags, each of which was programmed into a computer to identify individual heifers. The controlled feeding system reads the RFID tags to identify which heifers are permitted to eat from each of the feed rations. An automatic gate in each unit opens only when it identifies a heifer permitted to eat from it. The controlled feeding system was also programmed to restrict a heifer's time in the feeder to thirty (30) seconds so that other heifers could access the feeder. A gate rose to lift the heifer's head away from the feed when her time was up. Each heifer can return to the feeder later to finish eating their allotted amount. The gate cut off access to any animal with a full feed allocation. The computer also tracked how many times each heifer accessed the feeder and tracked each feeding behavior. Between meals in the automatic feeders, the heifers were free to graze from fescue-free pastures at the Savoy Unit, but forage primarily came from ad libitum access to hay.

Powdered melatonin (Cayman Chemical Company) was mixed into 100% ethanol and mixed with feed (fescue seed, corn gluten, and molasses) in a concrete mixer until the feed was dry (ethanol evaporates, and melatonin remains). Melatonin was dosed at about 100 μg/kg of body weight or about 50 mg/head/day for all groups. Ad libitum Bermuda grass hay was offered in an adjacent pasture area to encourage animals to leave the barn after consuming the feed ration during both the training and the feed trial period. After completion of the feed trial, heifers were maintained on fescue-free pastures at the Physiology Farm and supplemented as needed through calving and until their calves were weaned.

Heifers were weighed prior to the training period, prior to the feed trial period, and every fourteen (14) days until the end of the feed trial. Blood samples were collected from heifers via jugular venipuncture (two 10 mL purple top tubes) before the start of the feed trial and approximately every 28 days until the end of the feed trial. Doppler ultrasound of the uterine artery was conducted to quantify uterine artery blood flow prior to and at the end of the 70-day feed trial period.

Calf body weight was recorded at birth, and calves were tagged for identification purposes. Blood samples were collected from calves at 12±2 days of age via jugular venipuncture (four 10 mL purple top tubes and one 4 mL purple top tube). At weaning, body weight was recorded, and blood samples were collected via jugular venipuncture (four 10 mL purple top tubes and one 4 mL purple top tube). Vaccinations were administered to calves according to normal procedures at the Savoy Unit. Heifers and calves were maintained according to standard procedures at the Savoy Unit and the Physiology Farm.

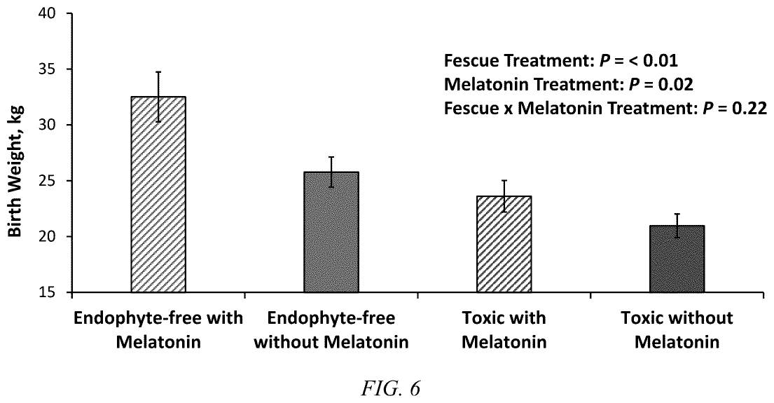

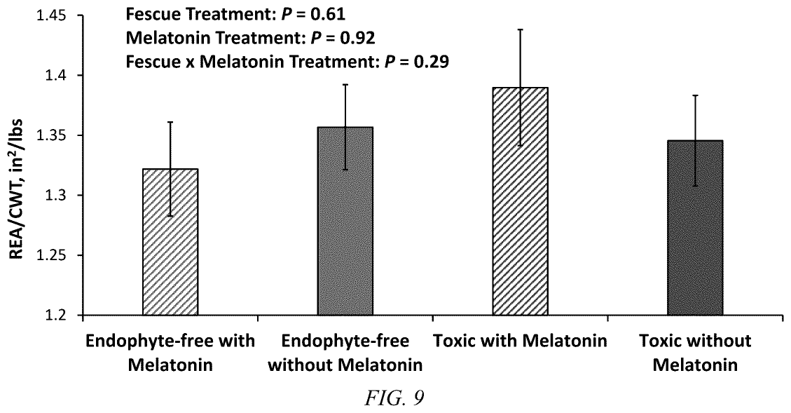

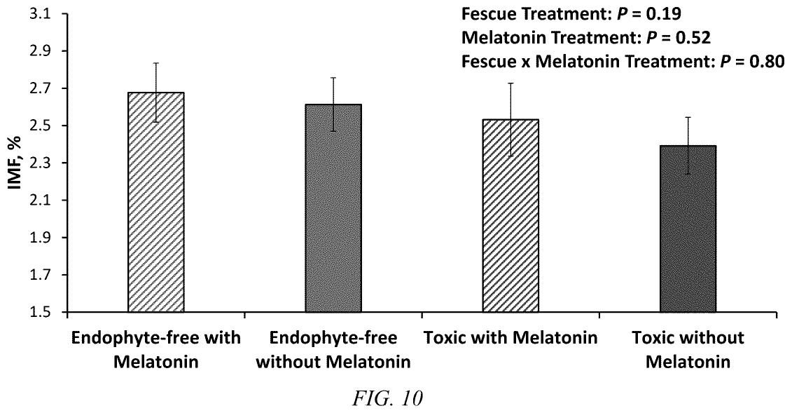

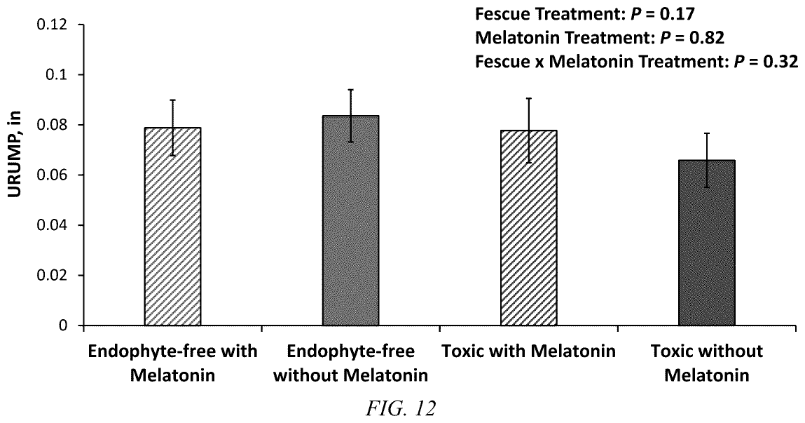

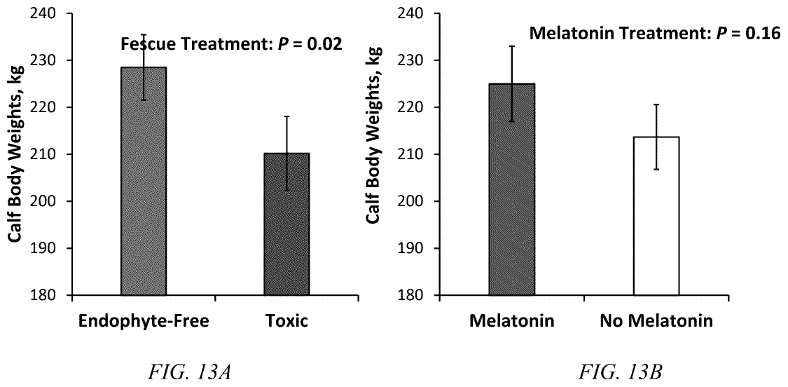

Calves were weaned at 226±19 d of age. Gestation length, calf birth weight, 205-day adjusted weaning weight (205-d WW), carcass ultrasonography measures (ribeye area, REA: REA/CWT), percent intramuscular fat (IMF), rib fat thickness (UFAT), rump fat thickness (URUMP), post-weaning weights (recorded every 28 days between weaning and 84 days post-weaning), and post-weaning average daily gain (ADG) (between weaning and 84 days post-weaning) were evaluated in offspring (n=44) (FIGS. 6-13). Post-weaning calf weights were analyzed using the MIXED procedure of SAS specific for repeated measures with treatment and time as fixed effects and replicate as random. All other variables were analyzed using the MIXED procedure of SAS with treatment as a fixed effect and replicate as random with gestation length as a covariate for birth weight. Table 1 summarizes the gestation length and body weights of calves born to primiparous dams that consumed E- or E+ fescue seed with (M) or without melatonin (NM) supplementation during mid-late gestation. Table 2 summarizes body composition ultrasonography measures for the same calves. Data in Tables 1 and 2 are presented as LS MEANS±SEM.

TABLE 1
Gestation Length and Body Weights.

| | Fescue | | Melatonin | | P-values | | |
|---|---|---|---|---|---|---|---|
| | Treatment Means | | Treatment Means | | | | Fescue × |
| Variable | E− | E+ | NM | M | Fescue | Melatonin | Melatonin |
| Gestation | 274.46 ± | 270.46 ± | 272.93 ± | 272.00 ± | 0.01* | 0.54 | 0.82 |
| Length (d) | 1.01 | 1.11 | 0.99 | 1.13 | | | |
| Birth | 28.10 ± | 21.50 ± | 23.69 ± | 25.90 ± | <0.01* | 0.07 | 0.68 |
| Weight (kg) | 0.93 | 1.02 | 0.90 | 1.02 | | | |
| 205-d WW | 180.66 ± | 164.83 ± | 168.54 ± | 176.95 ± | 0.03* | 0.23 | 0.03* |
| (kg) | 6.75 | 7.45 | 6.68 | 7.56 | | | |

*p-value < 0.05

TABLE 2
Body Composition Ultrasonography Measures.

| | Fescue | | Melatonin | | P-values | | |
|---|---|---|---|---|---|---|---|
| | Treatment Means | | Treatment Means | | | | Fescue × |
| Variable | E− | E+ | NM | M | Fescue | Melatonin | Melatonin |
| REA (in.²) | 5.90 ± | 5.54 ± | 5.57 ± | 5.87 ± | 0.21 | 0.30 | 0.03* |
| | 0.33 | 0.35 | 0.32 | 0.35 | | | |
| REA/cwt | 1.34 ± | 1.36 ± | 1.35 ± | 1.35 ± | 0.62 | 0.92 | 0.71 |
| (in.²/lbs.) | 0.03 | 0.03 | 0.03 | 0.03 | | | |
| IMF (%) | 2.65 ± | 2.46 ± | 2.50 ± | 2.60 ± | 0.21 | 0.54 | 0.98 |
| | 0.11 | 0.13 | 0.11 | 0.14 | | | |
| UFAT (in.) | 0.09 ± | 0.08 ± | 0.08 ± | 0.08 ± | 0.08 | 0.96 | 0.14 |
| | 0.01 | 0.01 | 0.01 | 0.01 | | | |
| URUMP | 0.08 ± | 0.07 ± | 0.08 ± | 0.08 ± | 0.45 | 0.60 | 0.14 |
| (in.) | 0.01 | 0.01 | 0.01 | 0.01 | | | |

*p-value < 0.05

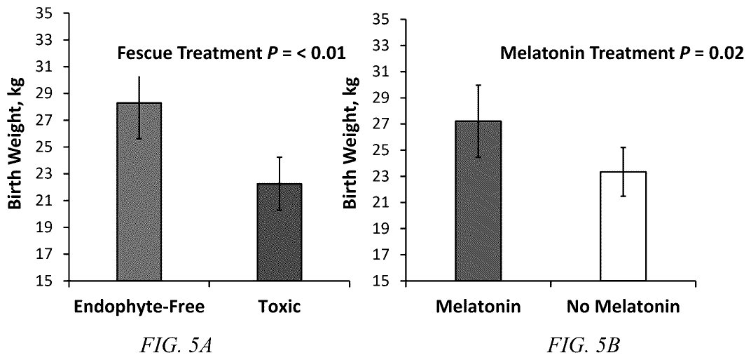

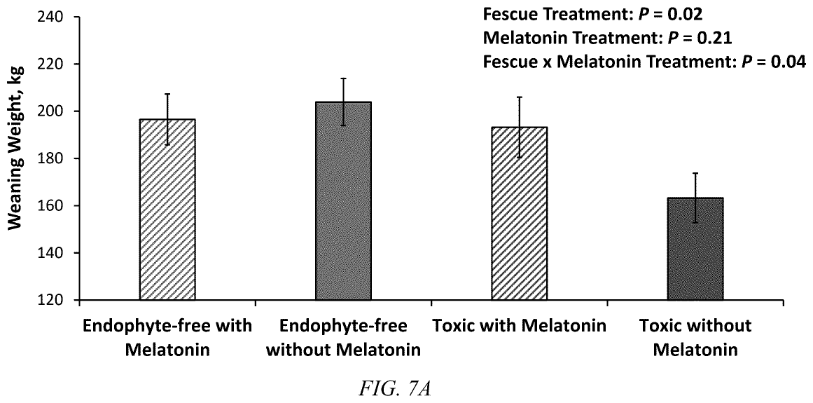

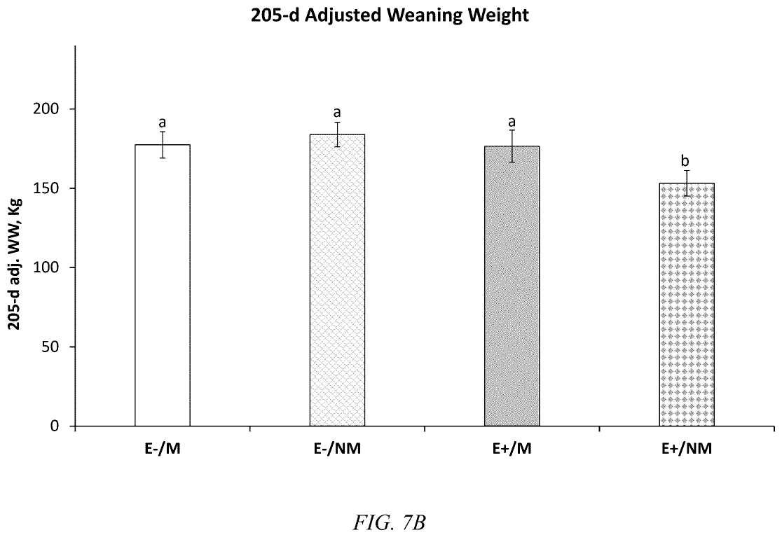

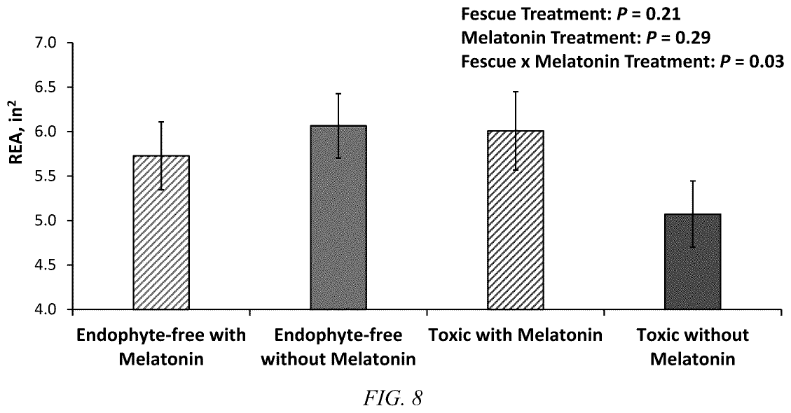

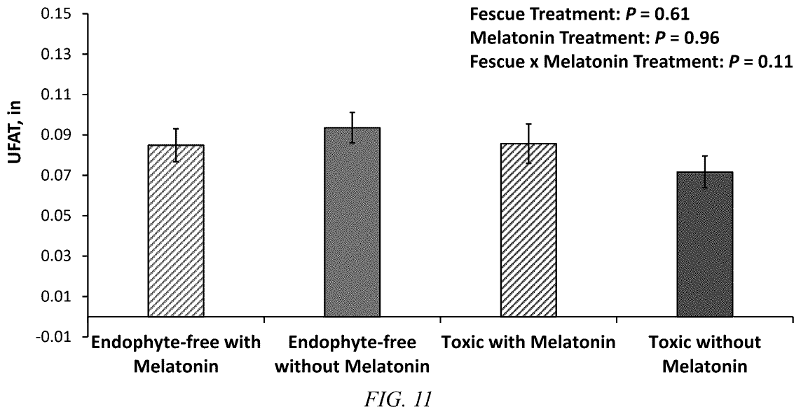

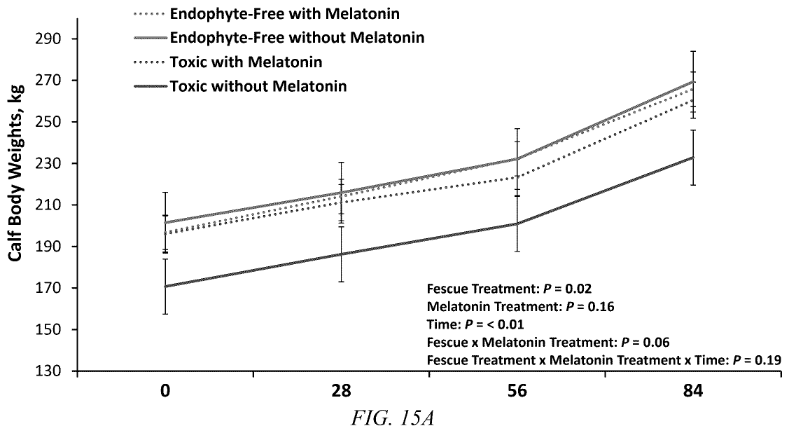

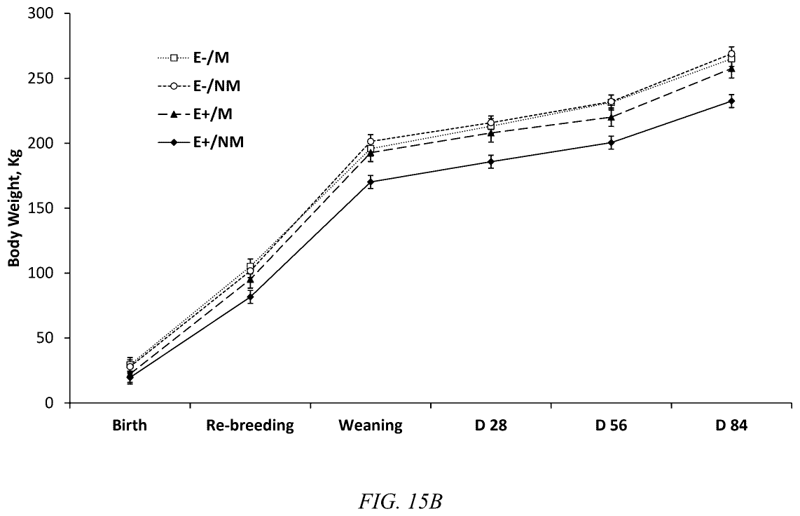

Gestation length, birth weight, 205-d WW, and post-weaning calf weight were decreased (P<0.04) in offspring of dams that consumed E+ relative to E− during gestation. As illustrated, birth weight was increased (P=0.02) in offspring of dams receiving melatonin relative to those without melatonin during gestation (FIGS. 5A, 5B, and 6; Table 1). The 205-d WW and REA were decreased in calves born to E+/NM dams relative to E−/NM. E−/M, and E+/M (P<0.05) (FIGS. 7 and 8). Post-weaning weight tended to be decreased in calves born to E+/NM dams relative to E−/NM, E−/M, and E+/M (P=0.06) (FIG. 15A), and when all time points were analyzed together, a treatment-by-time interaction showed a significant difference in body weight at and after weaning (FIG. 15B). UFAT tended to be decreased (P=0.08) in offspring of dams that consumed E+ relative to E− during gestation (Table 2; FIG. 11). There were no other effects of treatment, time, or interaction (P>0.10).

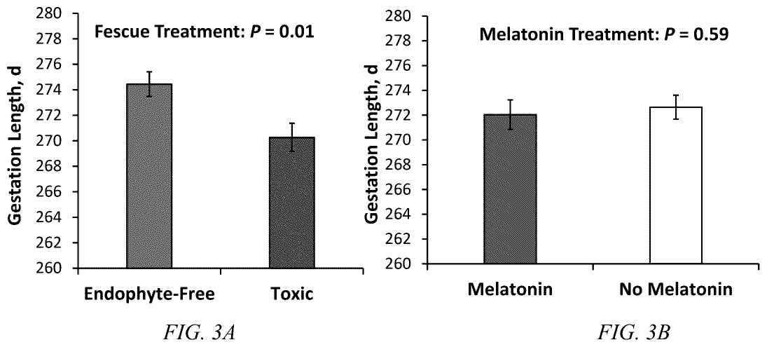

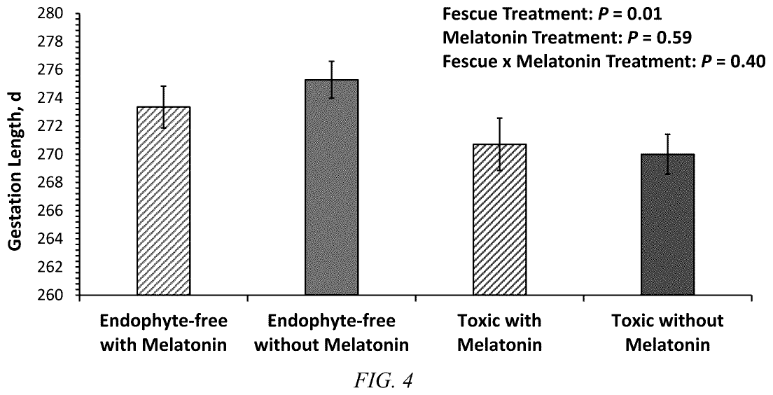

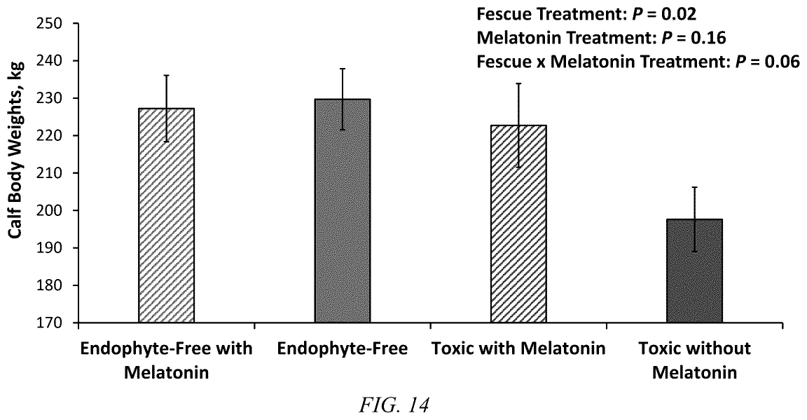

As can be seen in FIGS. 3-7 and 13-15, gestation length, birth weight, 205-d weaning weight, and post-weaning calf weight were decreased in calves born to dams that received toxic fescue seed relative to those receiving endophyte-free. Birth weight was increased in calves born to melatonin-supplemented dams relative to those without (FIGS. 5A, 5B, and 6). There was a fescue×melatonin treatment interaction, in which 205-d weaning weight, ribeye area, and post-weaning calf weight were increased in calves born to dams that received toxic fescue seed with melatonin compared to toxic fescue seed without melatonin (FIGS. 7-9, and 13-15). Notably, the group that received melatonin supplements in addition to toxic fescue seed was statistically similar to the endophyte-free groups.

The inventive process of melatonin treatment for pregnant livestock improves producer returns by weaning heavier calves, in addition to benefits extending through the stocker phase and feedlot phase of production. The therapeutic process of using melatonin in livestock consuming toxic fescue may also improve health, immune function, growth, performance, metabolic function, feed efficiency, milk production, microbiome, and reproductive traits. The therapeutic process may also improve the health, immune function, growth, performance, metabolic function, feed efficiency, milk production, microbiome, and reproductive traits of livestock whose dams consumed toxic fescue during gestation. Based on the foregoing, melatonin is an effective therapeutic treatment to improve the performance of offspring born to dams consuming toxic fescue, and it is a more cost-effective solution than replacing toxic fescue with a non-toxic alternative.

### Example 2: Uterine and Coccygeal Artery Hemodynamics

In particular, the objective of this example was to determine the influence of melatonin supplementation on uterine and coccygeal artery hemodynamics of pregnant beef cattle grazing toxic, endophyte-infected (E+) tall fescue during mid-late gestation.

Multiparous crossbred cows were artificially inseminated with sex-sorted semen (Y chromosome-bearing sperm) from a single Angus sire and maintained on pastures free from E+ fescue prior to the start of the treatment period. Pregnant cows (n=50) were assigned to one of three treatment groups based on body weight, with two replications per treatment: non-toxic, novel endophyte-infected (NE+) fescue without melatonin supplementation (NE+/NM; n=16), E+ fescue with melatonin supplementation (E+/M; n=16), and E+ fescue without melatonin supplementation (E+/NM; n=18). Treatments were administered over a 70-d period between May and July (d 147±1 to 217±1 of gestation). A commercially available feed was mixed with or without melatonin [100 μg/kg body weight (BW)] and hand-fed daily.

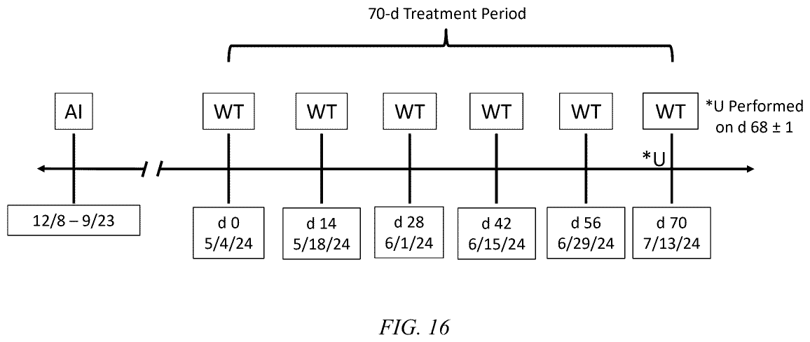

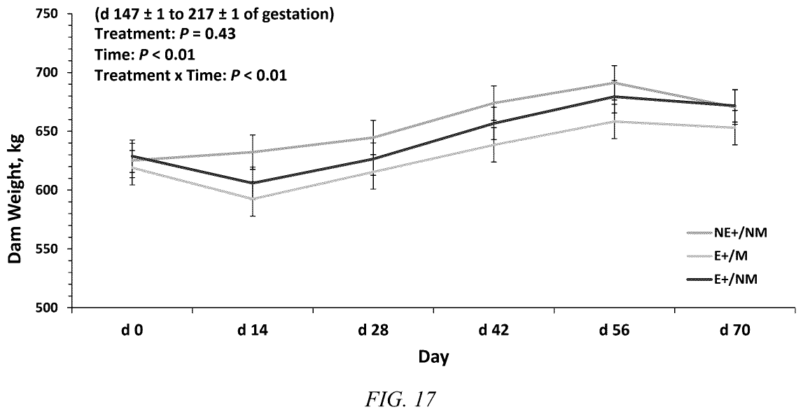

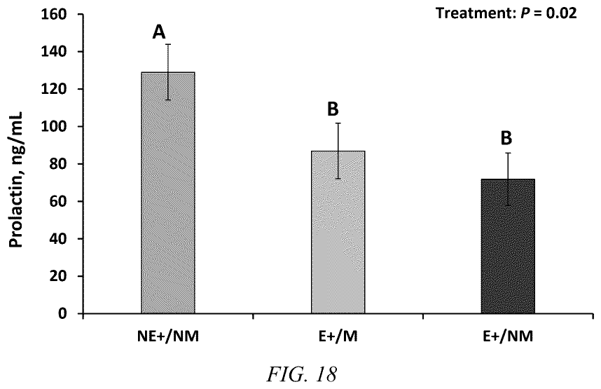

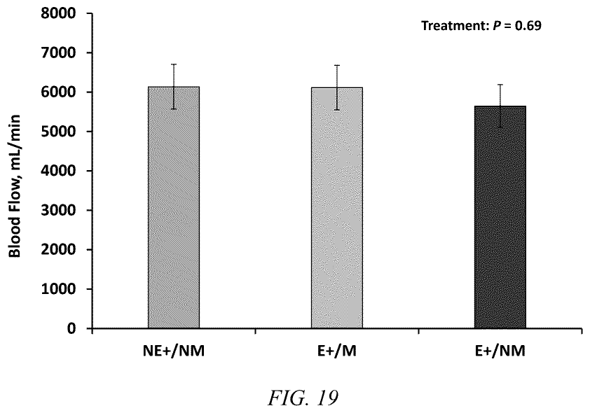

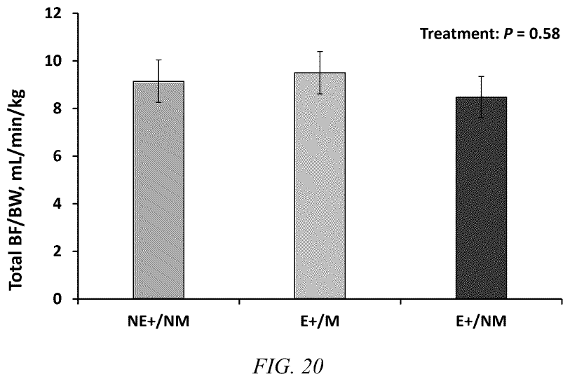

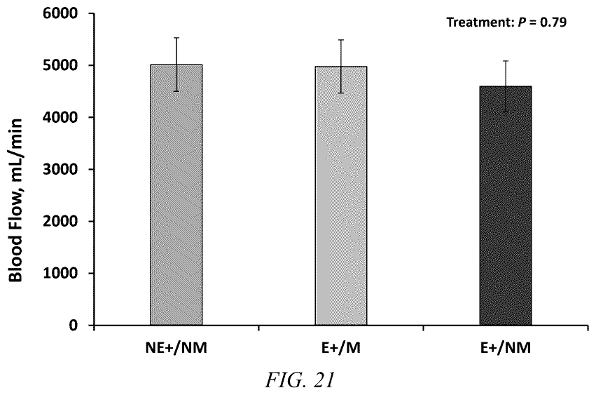

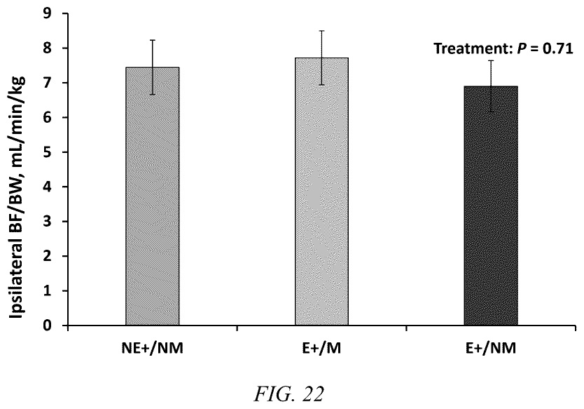

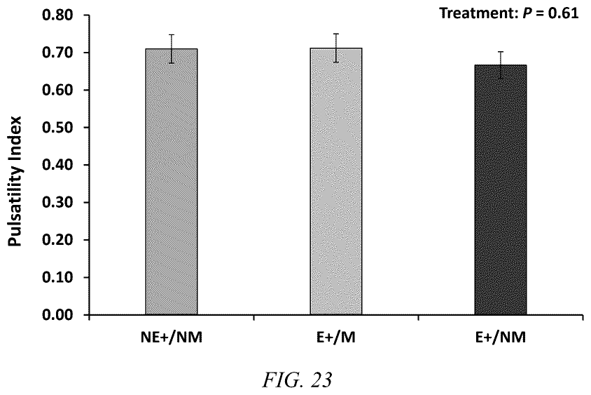

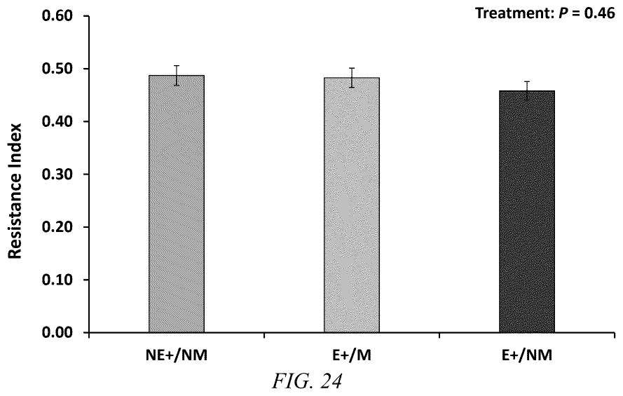

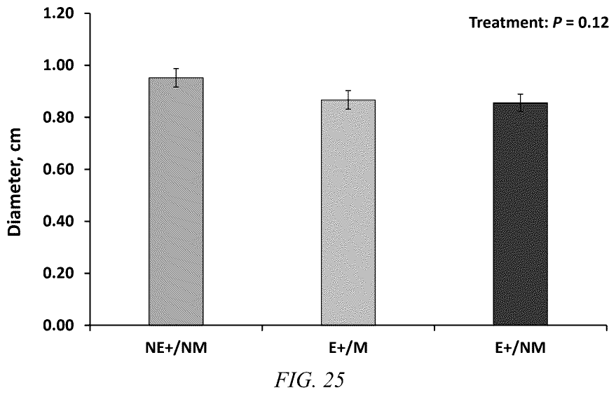

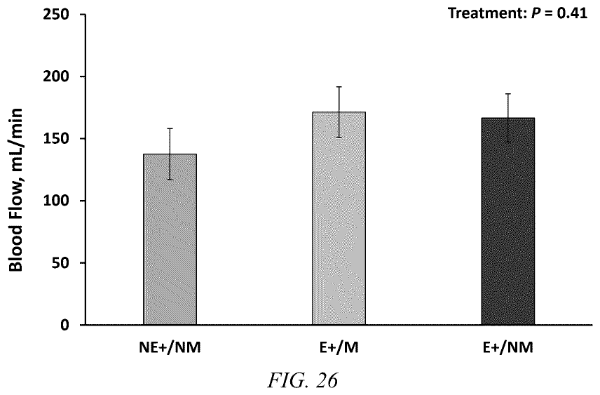

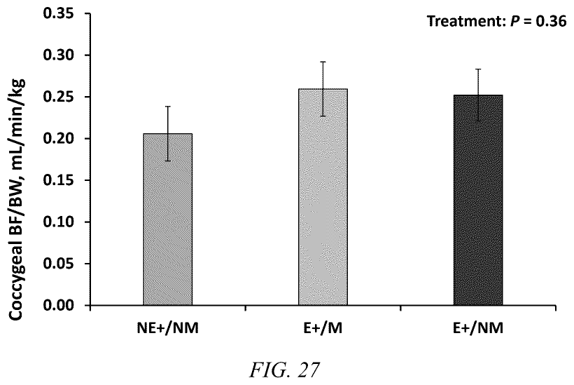

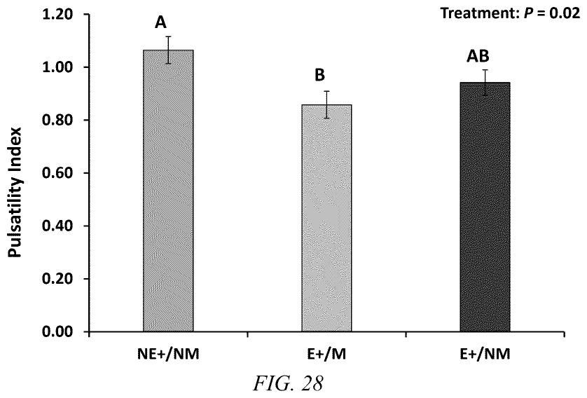

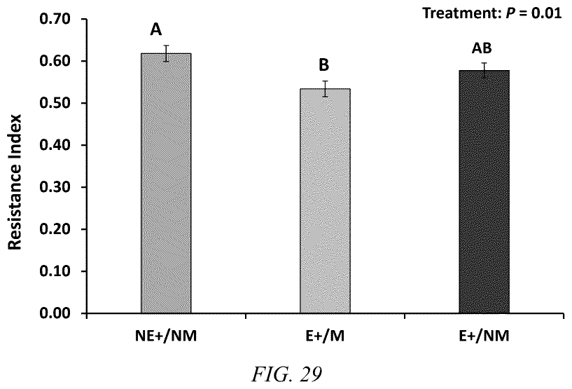

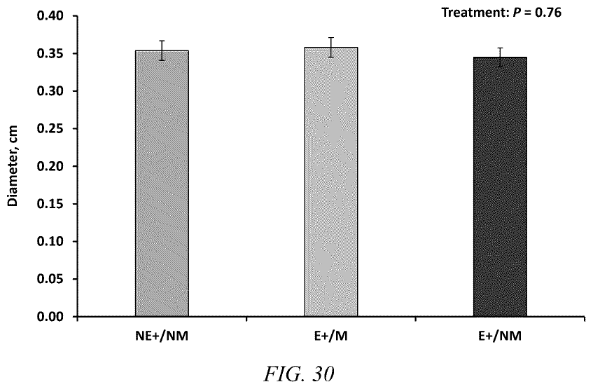

Cows were weighed every fourteen (14) days relative to the start of the 70-d treatment period (FIG. 16-17): i.e., at d 0, 14, 28, 42, 56, and 70. Measurements of plasma prolactin were obtained on d 70 of the treatment period by radioimmunoassay (FIG. 18). Color Doppler ultrasound of the uterine and coccygeal artery was conducted at d 68±1 of the treatment period. Cardiac cycle waveforms from three independent ultrasound scans were used to determine the following uterine artery measures (FIGS. 19-25) and coccygeal artery measures (FIGS. 26-30): diameter (cm), pulsatility index (PI), resistance index (RI), blood flow (BF; mL/min; equal to MnV*vessel area*60 s), and blood flow as a proportion of BW (BF/BW).

After the treatment period, cattle were maintained together as a single group on pastures that were free from E+ fescue. Body weights were analyzed using the MIXED procedure of SAS specific for repeated measures with treatment, time, and the interaction as fixed effects and replicate as random. Arterial hemodynamics and prolactin measures were analyzed using the MIXED procedure of SAS with treatment as a fixed effect and replicate as a random effect. Ultrasonography day and order were tested as covariates and removed from the model (P<0.05).

There was an effect of time and a treatment by time interaction on cow BW (P<0.01). Prolactin was decreased (P=0.02) in E+/NM (71.8±14.0 ng/mL) and E+/M (86.9±14.9 ng/mL) relative to NE+/NM (128.9±14.9 ng/mL) cows. Coccygeal artery PI was decreased (P<0.02) in E+/M (0.86±0.1) relative to NE+/NM (1.06±0.1) cows. Coccygeal artery RI was decreased (P<0.01) in E+/M (0.53±0.02) relative to NE+/NM (0.62±0.02) cows. There were no other effects of treatment (P>0.05).

These results suggest limited effects of melatonin on uterine and coccygeal artery hemodynamics in pregnant multiparous crossbred beef cows grazing endophyte-infected tall fescue.

### Example 3: Post-Calving Dam Weight, Milk Production, and Steer Preweaning Growth

In particular, the objective of this example was to evaluate the influence of melatonin supplementation in pregnant beef cattle grazing toxic, endophyte-infected tall fescue (E+) on post-calving dam body weight (BW), milk production, and preweaning growth of steer calves.

The same artificially inseminated cows were used in Example 3 that were used Example 2. As outlined above in Example 2, forty-nine (n=49) multiparous crossbred beef cows were artificially inseminated with sex-sorted semen (Y chromosome-bearing sperm) from a single Angus sire and maintained on pastures free from E+ fescue prior to the start of the treatment period. Pregnant cows were stratified into three treatment groups based on body weight, with two replications per treatment: non-toxic, novel endophyte-infected (NE+) tall fescue without melatonin supplementation (NE+/NM; n=15), E+ fescue with melatonin supplementation (E+/M; n=16), and E+ fescue without melatonin supplementation (E+/NM; n=18). The treatment period was over 70 d between May and July (d 147±1 to 217-1 of gestation). A commercially available feed was mixed with melatonin (100 μg/kg BW) or without melatonin and hand-fed to each cow daily. At the conclusion of the treatment period (56±18 d before parturition), cattle were moved to a single group and maintained on pastures free from E+ fescue, where they remained through weaning.

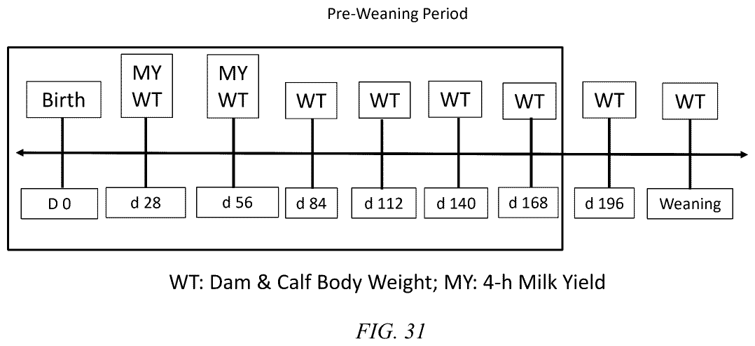

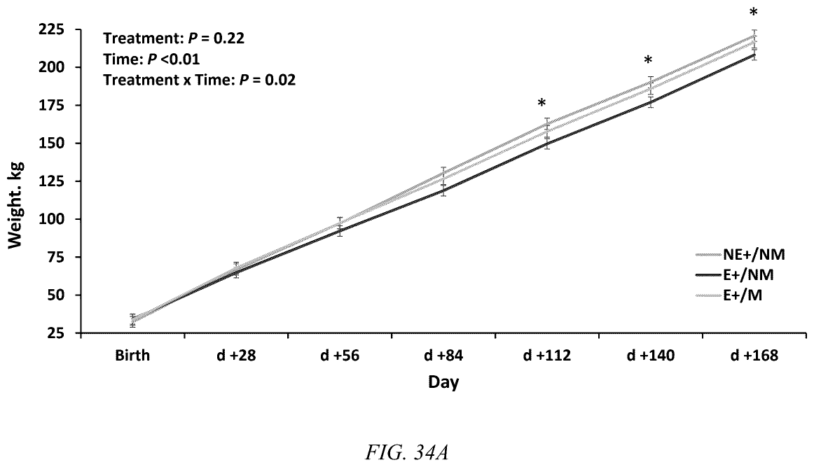

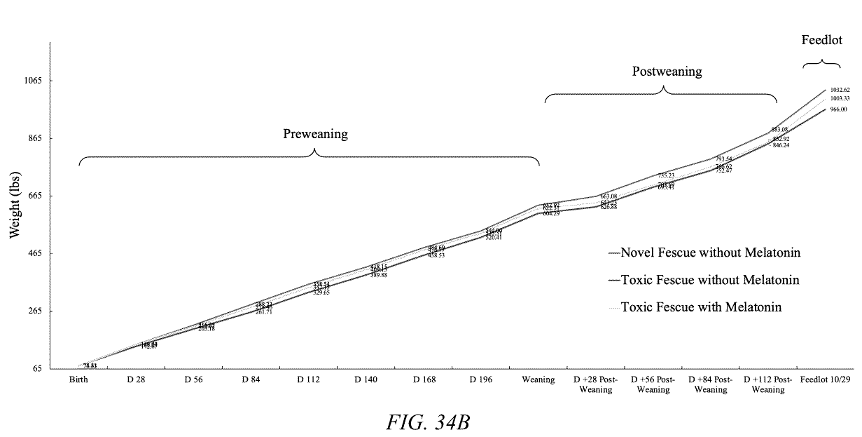

After birth, calves were castrated within 48-72 hours. Dams and steer calves were weighed every 28 days relative to calving (FIG. 31): i.e., at d 0, 28, 56, 84, 112, 140, and 168 (d 0=calving, <24 h). Table 3 depicts calf weight at preweaning, postweaning, and feedlot for NE+/NM, E/+M, and E/+NM (respectively, green, yellow, and red in FIG. 34B). Four-hour milk yields on d 28±3 and d 56±3 were used to estimate daily milk volume and weight. Protein, lactose, and triglycerides were evaluated in milk samples collected on d 28 and d 56.

TABLE 3
Calf weight (lbs.)

| | NE+/NM | E+/M | E+/NM |
|---|---|---|---|
| Birth | 75.31 | 74.43 | 75.31 |
| D 28 | 149.54 | 148.07 | 142.67 |
| D 56 | 216.23 | 212.07 | 203.18 |
| D 84 | 288.23 | 278.46 | 261.71 |
| D 112 | 358.54 | 347.15 | 329.65 |
| D 140 | 418.15 | 409.15 | 389.88 |
| D 168 | 484.69 | 476.77 | 458.53 |
| D 196 | 544.00 | 535.31 | 520.41 |
| Weaning | 632.92 | 622.31 | 604.29 |
| D +28 Post-Weaning | 663.08 | 641.23 | 626.88 |
| D +56 Post-Weaning | 735.23 | 703.69 | 695.41 |
| D +84 Post-Weaning | 793.54 | 766.62 | 752.47 |
| D +112 Post-Weaning | 883.08 | 852.92 | 846.24 |
| Feedlot - October 29 | 1032.62 | 1003.33 | 966.00 |

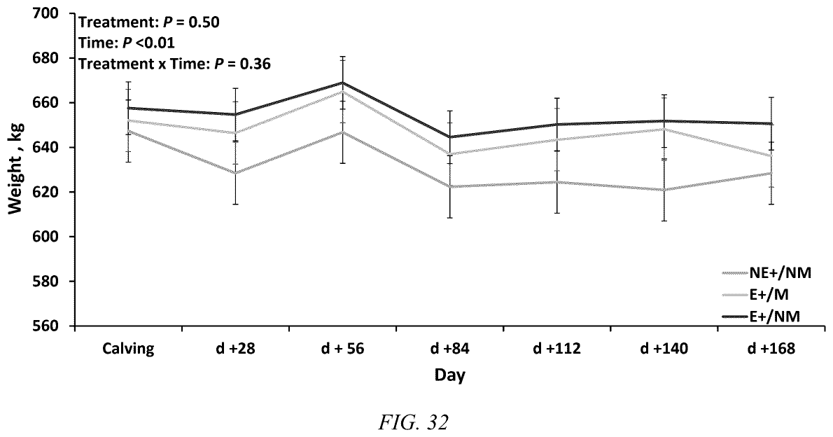

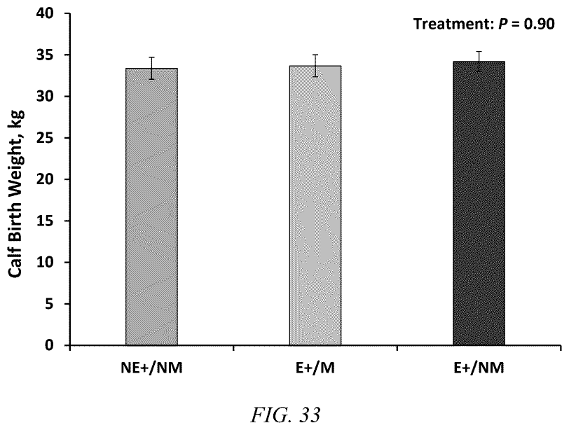

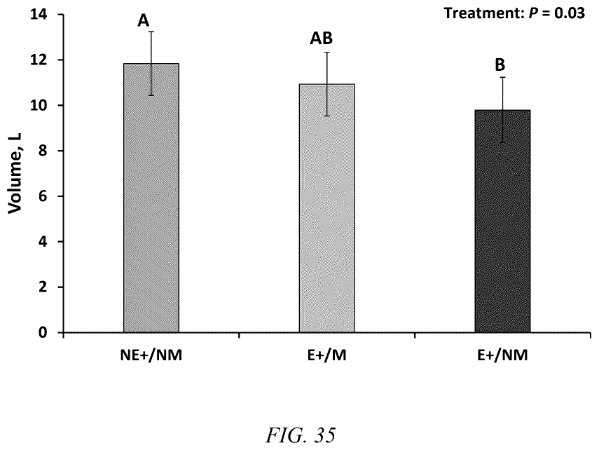

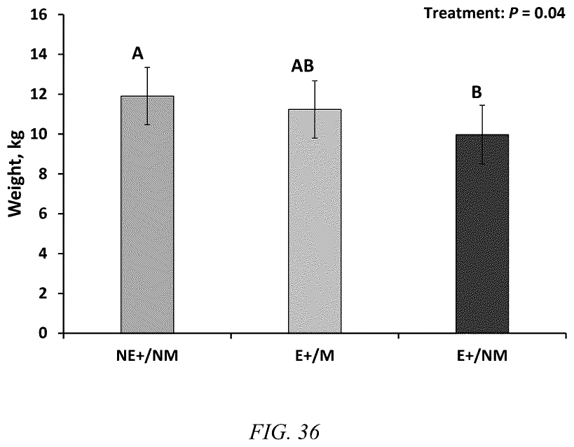

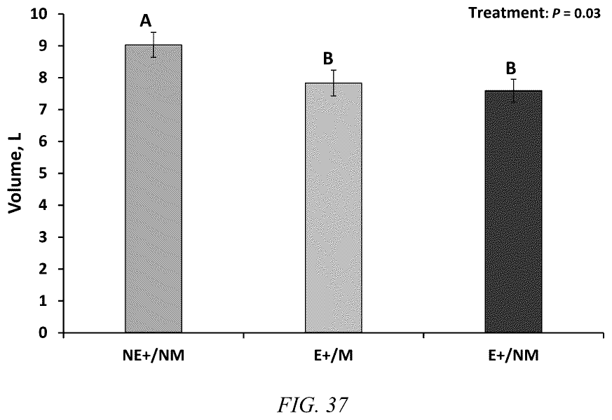

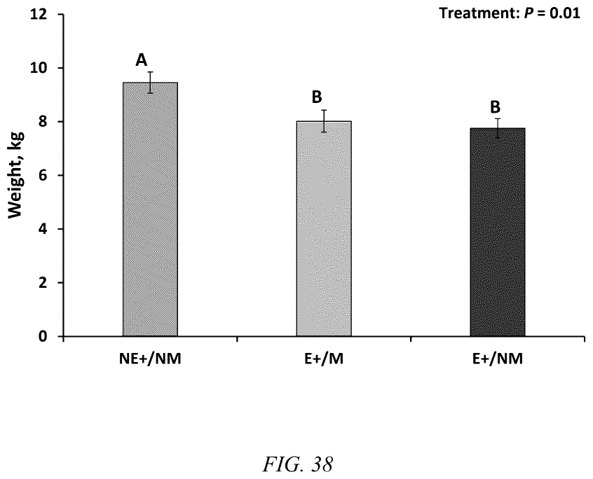

Dam body weight (FIG. 32) and calf body weight (FIG. 34) were analyzed, using the MIXED procedure of SAS specific for repeated measures with treatment and time as fixed effects and replicate as a random effect. Dam prolactin, calf birth weight (FIG. 33), milk volume (FIGS. 35 and 37), and milk weight (FIGS. 36 and 38) were analyzed using the MIXED procedure of SAS with treatment as a fixed effect and replicate as a random effect.

On d 28, estimated daily milk volume and weight were decreased (P<0.05) in E+/NM dams relative to NE+/NM dams. On d 56, estimated daily milk volume and weight were decreased (P<0.03) in E+/NM and E+/M dams relative to NE+/NM dams. There was an effect of time for dam and calf weights (P<0.01). There was a treatment by time interaction for calf body weight, with calves born to E+/NM dams having decreased body weight relative to calves born to NE+/NM and E+/M dams from d 112 to d 168 (P=0.02). There were no other effects of treatment, time, or the interaction (P>0.05).

These data suggest melatonin supplemented to pregnant crossbred multiparous beef cows grazing endophyte-infected tall fescue during mid-late gestation may increase pre-weaning growth of steer calves. It is contemplated that milk production may be a mechanism that is driving differences in postnatal growth for calves. Melatonin may “prime” the mammary gland to increase milk production when specifically fed during gestation.

### Discussion of Examples

Several key differences between Example 1 and Examples 2 and 3 are worthy of mention. Unlike Example 1, which used first-calf heifers as the mothers consuming toxic fescue seed and all-female offspring. Examples 2 and 3 used multiparous cattle grazing toxic fescue pastures and used all-male offspring. In Example 1, offspring born to heifers that consumed toxic fescue seed during a 70-day window in mid-late gestation were approximately 68 lbs. lighter at weaning than those born to mothers that consumed nontoxic seed during the same window of gestation. However, by feeding melatonin during that same 70-day window of fescue exposure, over 75% of that loss (approximately 53 lbs.) was recovered in calf weight at weaning. In the second study, a similar percentage recovery rate of the loss in weaning weights was found, but on a smaller scale. Specifically, 28 days prior to weaning, calves born to mothers that grazed toxic fescue during gestation were on average 28 lbs. lighter than calves born to mothers that grazed novel (a nontoxic variety) fescue. However, by feeding melatonin during the same window of time the pregnant mothers were grazing toxic fescue we were able to recover over 65% of that loss (approximately 19 lbs.). These numbers reflect are statistically adjusted means rather than raw group averages before statistical analysis).

Each of Examples 1-3 suggest a strong potential for melatonin to be used as a cost effective therapeutic to improve weaning weights of calves born in the fescue belt. Results suggest the potential to wean calves that are on average 53 lbs. heavier (when the mother is a first calf heifer) or 19 lbs. heavier (when the mother is a multiparous cow). Multiplied across an entire herd, this treatment could provide opportunities for significantly increased return on investment for cattle producers in the fescue belt.

Where applicable, although state diagrams, flow diagrams, or both may be used to describe embodiments, the invention is not limited to those diagrams or to the corresponding descriptions. For example, flow need not move through each illustrated box or state, or in exactly the same order as illustrated and described.

Processes of the disclosure may be implemented by performing or completing manually, automatically, or a combination thereof, selected steps or tasks.

The term “process” may refer to methods, manners, means, techniques, and procedures for accomplishing a given task including, but not limited to, those manners, means, techniques and procedures either known to, or readily developed from known manners, means, techniques and procedures by practitioners of the art to which the invention belongs.

It should be noted that where reference is made herein to a process comprising two or more defined steps, the defined steps can be carried out in any order or simultaneously (except where context excludes that possibility), and the process can also include one or more other steps which are carried out before any of the defined steps, between two of the defined steps, or after all of the defined steps (except where context excludes that possibility).

For purposes of the instant disclosure, the term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

Thus, the invention is well adapted to carry out the objects and attain the ends and advantages mentioned above as well as those inherent therein. While the inventive concept has been described and illustrated herein by reference to certain illustrative embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A composition for treating livestock that consumes toxic forage, the composition comprising:
an active ingredient comprising melatonin having a concentration of up to about 250 μg/kg body weight of the livestock; and
an agriculturally acceptable carrier.

2. The composition of claim 1 wherein the melatonin has a concentration of about 50 μg/kg to about 200 μg/kg body weight of the livestock.

3. The composition of claim 2 wherein the melatonin has a concentration of about 100 μg/kg body weight of the livestock.

4. The composition of claim 1 wherein the livestock is selected from the group consisting of bovine, sheep, goats, lambs, pigs, horses, or poultry.

5. The composition of claim 4 wherein the livestock comprises bovine that consumes toxic forage.

6. The composition of claim 5 wherein the bovine is cattle.

7. The composition of claim 6 wherein the cattle is a gestating dam that consumes toxic forage.

8. The composition of claim 1 wherein the toxic forage is selected from the group consisting of endophyte-infected tall fescue or ryegrass.

9. The composition of claim 8 wherein the toxic forage is endophyte-infected tall fescue.

10. The composition of claim 1 wherein the carrier is selected from the group consisting of a veterinary composition, an animal supplement, or a food composition.

11. The composition of claim 10 wherein the carrier is animal feed.

12. The composition of claim 10 wherein the carrier is a transdermal implant.

13. The composition of claim 10 wherein the carrier is a bolus dose.

14. The composition of claim 1 wherein the livestock is cattle, the melatonin has a concentration of about 100 μg/kg body weight of the cattle, and the toxic forage is endophyte-infected tall fescue.

15. A process for treating livestock that consumes toxic forage, the process comprising the steps of:
administering an active ingredient comprising melatonin having a concentration of up to about 250 μg/kg body weight of the livestock and an agriculturally acceptable carrier to the livestock that consumes toxic forage.

16. The process of claim 15 further comprises the step of administering the active ingredient comprising melatonin to the livestock as a prophylactic or therapeutic for the livestock that consumes toxic forage.

17. The process of claim 16 further comprises the step of administering the active ingredient comprising melatonin to the livestock at a predetermined time of day as the prophylactic or therapeutic for the livestock that consumes toxic forage.

18. The process of claim 15 further comprises the step of administering the active ingredient comprising melatonin to the livestock as a prophylactic or therapeutic for offspring of the livestock, wherein the livestock is a gestating dam that consumes toxic fescue.

19. The process of claim 18 further comprises the step of administering the active ingredient comprising melatonin to the livestock at a predetermined time of day as the prophylactic or therapeutic for offspring of the livestock.

20. The process of claim 15 wherein the melatonin has a concentration of about 50 μg/kg to about 200 μg/kg body weight of the livestock.

21. The process of claim 20 wherein the melatonin has a concentration of about 100 μg/kg body weight of the livestock.

22. The process of claim 15 wherein the livestock is selected from the group consisting of bovine, sheep, goats, lambs, pigs, horses, or poultry.

23. The process of claim 22 wherein the livestock comprises bovine that consumes toxic forage.

24. The process of claim 23 wherein the bovine is cattle.

25. The process of claim 24 wherein the cattle is a gestating dam that consumes toxic forage.

26. The process of claim 15 wherein the toxic forage is selected from the group consisting of endophyte-infected tall fescue or ryegrass.

27. The process of claim 26 wherein the toxic forage is endophyte-infected tall fescue.

28. The process of claim 15 wherein the carrier is selected from the group consisting of a veterinary composition, an animal supplement, or a food composition.

29. The process of claim 28 wherein the carrier is animal feed.

30. The process of claim 28 wherein the carrier is a transdermal implant.

31. The process of claim 28 wherein the carrier is a bolus dose.

32. The process of claim 15 further comprises the step of administering the active ingredient comprising melatonin having a concentration of about 100 μg/kg body weight to the livestock, wherein the livestock is a gestating dam that consumes endophyte-infected tall fescue.
